(19) United States
(12) Patent Application Publication
(10) Pub. No.: US 2008/0287767 A1
(43) Pub. Date: Nov. 20, 2008

(54) Biometric Sensor

(76) Inventors: Willem Franke Pasveer, Eindhoven (NL); Martin Ouwerkerk, Eindhoven (NL); Jim T. Oostveen, Eindhoven (NL)

Correspondence Address: PHILIPS INTELLECTUAL PROPERTY & STANDARDS, P.O. BOX 3001, BRIARCLIFF MANOR, NY, 10510, US

(21) Appl. No.: 12/094,671

(22) PCT Filed: Nov. 21, 2006
(86) PCT No.: PCT/IB06/54360

§ 371 Date: May 22, 2008

(30) Foreign Application Priority Data
Nov. 25, 2005 (EP) 05111277.9

(51) Int. Cl. A61B 5/0408

(52) U.S. Cl. 600/372

(57) ABSTRACT

A bio metric sensor device (1) comprises: —a thin, flexible, layered sensor body (2); —a conductive sense plate (21); —a first non-conductive layer (41) between the sense plate and an outer surface (7); —a conductive shield plate (53) having a passage opening (54), overlaying the sense plate; —a second non-conductive layer (51) between the sense plate and the shield plate; —conductive circuit lines (73) on an inner surface (6); —a non-conductive separation layer (61, 71) between the shield plate and the circuit lines; —a signal processing circuit (100) mounted on the inner surface (6), the circuit (100) comprising a differential amplifier (110) having an input (111); —a conductive interconnector (82) crossing the second non-conductive layer (51) and the separation layer (61, 71), extending through the passage opening (54) of the shield plate (53), coupling the sense plate (21) and said input (111) of said amplifier (110).

### FIELD OF THE INVENTION

The present invention relates in general to a biometric sensor for sensing bioelectrical signals.

### BACKGROUND OF THE INVENTION

It is commonly known that electrical signals are generated on various places of the human body, these signals being representative for electrical activity inside the human body. Important sources of such electrical activity are the heart, the brain, moving muscles, etc. It is already known to measure these electrical signals, and to provide a time-registration of these signals such as for instance an electrocardiogram (ECG), an electro-encephalogram (EEG), an electro-myogram (EMG), in order to obtain information regarding certain body conditions.

When measuring these signals, some problems have to be overcome. A first problem relates to the fact that the human skin is a poor conductor. In this context, measuring sensors can be classified as follows. A penetrating sensor, for instance a needle, penetrates the skin and will have a good electrical contact with the conductive parts of the body below the skin, but such sensors are not suitable in practical situations. Contact electrodes, in the form of a conductive plate placed in close contact with the skin, suffer from the relatively high contact resistance between the sensor and the skin. In order to reduce this problem by improving the galvanic contact, wet electrodes are used, comprising a conducting gel (containing silver chloride) between the conductive plate and the skin; however, this gel can cause irritations or even allergic reaction.

In order to overcome the above-mentioned problems and disadvantages of contact electrodes, contact less sensors have already been developed for measuring the electrical signals by a capacitive coupling. However, such capacitive sensors introduce problems of a different kind. The most important problems in this respect are related to the fact that such capacitive sensors are also sensitive to electrical signals generated by the surroundings. Important sources of disturbance signal or noise signals are the electrical mains wiring (carrying voltages in the order of 230 V or more) or moving bodies which are charged electrostatically to a high voltage (which may be in the order of 1000 V or more).

A second problem relates to comfort for the user. In practice, capacitive biometric sensors have already been proposed which are rigid and relatively heavy. Although biometric sensors have several possible applications, one important field of application is implementation in and or integration with clothing. In such applications, rigid sensors are undesirable, because they are not comfortable for the user. Further, rigid sensors have the problem of providing only poor contact with the skin: for a good contact, it is required that the biometric sensor has sufficient flexibility to adapt to the curvature of the body and to follow changes in this curvature, for instance in the case of moving muscles.

The same types of problems are encountered when such sensors are implemented in the surface material of a chair, or a bed, or an examination table, allowing to easily obtain body-signals of a person without having to specifically apply sensors to the skin of that person.

International patent publication WO 2005/032368 discloses a flexible biometric sensor, which provides a capacitive coupling with the skin. The sensor of this publication comprises a conductive cloth, provided by incorporating conductive wires in a textile material. A disadvantage of such design is that it requires an adaptation to the textile manufacturing process. A further disadvantage is that such cloth will typically cover a relatively large surface area, so that the spatial resolution of the sensor is relatively low. Conversely, if a cloth with a relatively small surface area would be used, such sensor would contain only a low number of conductive wires, providing only a poor coupling with the signals to be detected.

On the other hand, such sensor will be quite sensitive for signals from the surroundings, and it will be very difficult to discriminate between actual body signal and noise signals. In this respect it is worth noting that the noise signals may have amplitudes in the order of 100 mV or more, whereas the actual body signals may have amplitudes in the order of 1 mV or less.

The present invention aims to overcome the above-mentioned problems and disadvantages.

Specifically, the present invention aims to provide a biometric sensor device that has sufficient flexibility for adaptation to the curvature of the human body, is suitable for incorporation in clothing, and has reduced sensitivity for electrical signals from the surroundings.

### SUMMARY OF THE INVENTION

According to an important aspect of the present invention, a biometric sensor device comprises a stack of flexible, conductive layers, separated from each other by flexible insulating layers. A first layer comprises a sensing area. A second layer comprises a guard plate. The device further comprises integrated signal processing circuitry, and a further conductive layer, connected to a predetermined voltage level, preferably zero voltage, covering the electrical circuit.

### BRIEF DESCRIPTION OF THE DRAWINGS

These and other aspects, features and advantages of the present invention will be further explained by the following description of a preferred embodiment of the sensor device according to the present invention with reference to the drawings, in which same reference numerals indicate same or similar parts, and in which:

FIG. 1A schematically shows a top view of a biometric sensor device according to the present invention;

FIG. 1B schematically shows a top view of the biometric sensor device of FIG. 1A from the opposite direction;

FIG. 2 is a schematic cross section of a part of a flexfoil;

FIG. 3 is a schematic cross section of the biometric sensor device of FIG. 1A;

FIGS. 4A-C schematically illustrate steps in a possible manufacturing process for manufacturing the biometric sensor device of FIG. 1A;

FIG. 5 is a block diagram of an electronic signal processing circuit of the biometric sensor device of FIG. 1A.

### DETAILED DESCRIPTION OF THE INVENTION

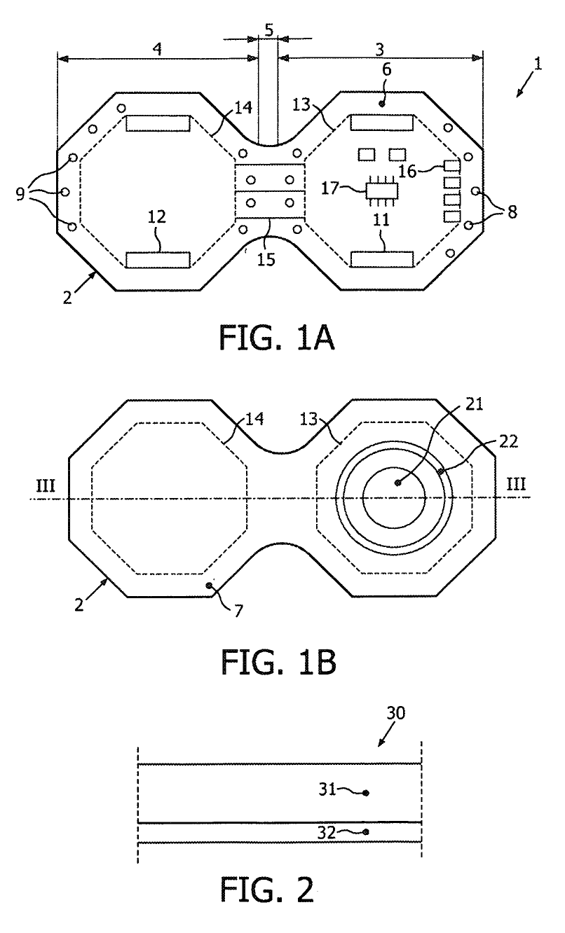

FIG. 1A is a schematic inside view of a preferred embodiment of a biometric sensor device 1 according to the present invention, and FIG. 1B is a schematic outside view of the same device. The sensor device 1 comprises a thin, flexible sensor body 2, comprising two wing parts 3, 4 attached to each other at a fold portion 5. The sensor body 2 has two opposite main surfaces, i.e. a first main surface 6 visible in the inside view of FIG. 1A, and an opposite second main surface 7, visible in the outside view of FIG. 1B. In use, the two wing parts 3, 4 will be folded together, such that the fold portion 5 takes the shape of a loop, and the first main surfaces 6 of the two wing parts will be facing each other; for this reason, the first main surface 6 will also be indicated as “inside surface”, whereas the opposite second main surface 7, which will be on the outside of the device when folded as mentioned, will also be indicated as “outside surface”.

The shape of the contour of the two wing parts 3, 4 is not essential. Typically, these two wing parts will have identical contours, but even that is not essential. In the illustrated embodiments, the two wing parts have an octagonal contour, but other contours, such as a circular contour, are also possible.

The first wing part 3 has a first series of through holes 8 along its perimeter; likewise, the second wing part 4 has a second series of through holes 9 along its perimeter. The first holes 8 and the second holes 9 are located such that, when the two wing parts 3 and 4 are folded together, the first holes 8 and the second holes 9 are aligned with each other. These holes facilitate the sensor device 1 being attached to clothing, for instance by stitches.

As will be explained in more detail below, the first wing part 3 has, on its outside surface 7, a substantially centrally located, electrically conductive sense plate 21 and an annular, electrically conductive guard ring 22 arranged around the sense plate 21. The shape of the sense plate 21 is not critical, but a circular shape is preferred. Likewise, the shape of the guard ring 22 is not critical, but a circular shape is preferred for the guard ring as well. The diameter of the sense plate 21 is not critical, and typically is a trade-off between on the one hand positional accuracy and on the other hand electrical sensitivity. In a suitable embodiment, the diameter of the sense plate 21 will typically be in the range from 10 to 15 mm, and an experimental embodiment has a diameter of 12 mm. The guard ring 22 may typically have a width in the order of 1 to 2 mm, and the radial distance between the sense plate 21 and the guard ring 22 may also typically be in the range of 1 to 2 mm.

As visible in the inside view of FIG. 1A, the first wing part 3 carries contact pads 16 for attaching external lines, and electronic circuit components 17.

As indicated by dotted lines, the sensor body 2 also comprises a first ground plate 13 in the first wing part 3, and a second ground plate 14 in the second wing part 4. These ground plates 13, 14, which are both electrically conductive yet thin enough to be mechanically flexible, are located at a distance from the inside surface 6 and at a distance from the outside surface 7, and are therefore shown in dotted lines in FIGS. 1A and 1B.

As shown in FIG. 1A, the first wing part 3 has, at its inside surface 6, at least one electrically conductive contact region 11, which is electrically connected to the first ground plate 13. Likewise, the second wing part 4 has at least one electrically conductive contact 12, which is electrically connected to the second ground plate 14. In the embodiment shown, the first wing part 3 has two contacts 11 located diametrically opposite to each other, and the same applies to the second wing part 4. The contacts 11 and 12 are located such that, when the sensor body is folded, the contacts 11 and 12 of the two wing parts 3 and 4 are aligned with each other. Thus, these contacts assure an electrical connection between the first ground plate 13 and the second ground plate 14. The contacts may also be used for mechanically sealing the sensor body 2 in its folded condition. In a possible embodiment, the contacts 11, 12 may be provided with a solder tin, and a local heat treatment after folding the sensor body 2 may cause the opposite contacts 11, 12 to be soldered to each other.

In the following, a more detailed description of the internal design of the sensor body 2 will be given.

First, reference is made to FIG. 2, schematically showing a cross section through a flexible foil 30, commonly known as “flex foil”, and comprising a first layer 31 and a second layer 32. The first layer 31 is electrically substantially non-conductive, and the second layer 32 is electrically substantially conductive. Typically, the second layer 32 is a thin copper layer, having a thickness in the order of about 10 to 20 μm. In a standard available product, this thickness is about 17.5 μm, in another standard available product this thickness is about 35 μm. The second layer is an insulator with the electric properties. A typical material for the non-conductive first layer 31 is capton. A flex foil 30 of the design of FIG. 2 is commercially available, in different sizes of the thickness of the non-conductive first layer 31, and this product is typically used as so-called “flexible PCB”. Since this material is known per se, as will be clear to a person skilled in the art, a further description is not needed here. However, it is noted that such commonly known flex foil 30 can be used in manufacturing the sensor body 2, more particularly by attaching multiple layers of flex foil 30 on top of each other, as will be clear from the following description. Attaching can be done by using a suitable adhesive, or by performing a heat treatment causing the capton layers to flow and adhere to neighbouring layers.

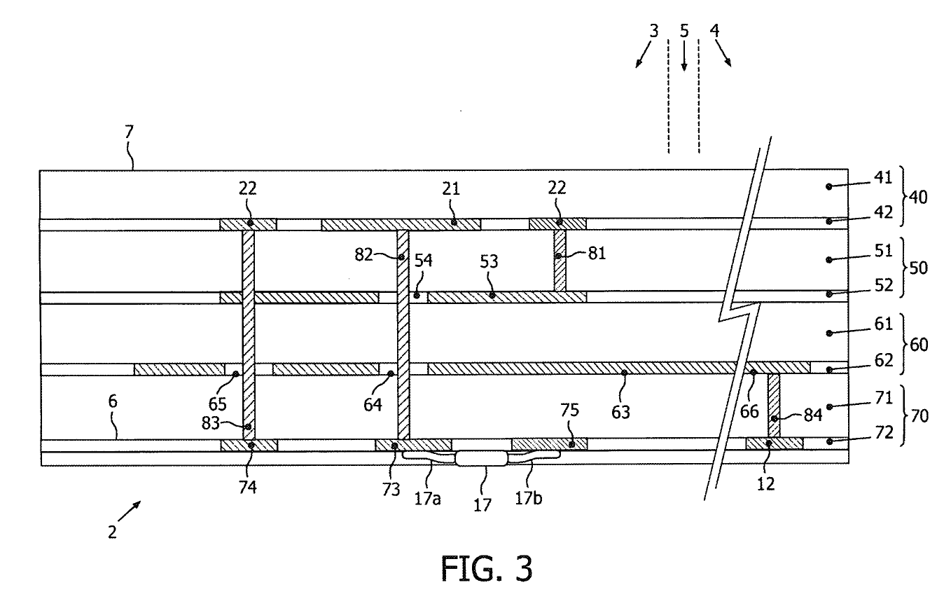

FIG. 3 schematically shows, not to scale, a cross section of the biometric sensor device 1 along the line III-III in FIG. 1B. In this embodiment, the sensor body 2 is comprised of a stack of four flex foil layers 40, 50, 60, 70 attached on top of each other. The first flex foil layer 40 has its non-conductive layer 41 directed to the outside of the device, such that this first non-conductive layer forms the outside surface 7 of the sensor device 1. Using commonly known techniques, such as etching, a part of the second conductive layer 42 has been removed, leaving the conductive sense plate 21 and the conductive annular guard ring 22 around the sense plate 21.

In use, the sensor device 1 may be brought in close proximity to the skin of a human body to be examined, and may even be brought in contact with this body. Then, the non-conductive layer 41 will act as an electric insulator, providing a galvanic insulation between the body and the conductive sense plate 21, and also acting as a dielectricum between the human body and the sense plate 21. Thus, the sense plate 21 will pick up variations in the electrical field present in the human skin.

The second flex foil 50 is attached to the first flex foil 40, such that the second non-conductive layer 51 is in contact with the first conductive layer 42. Effectively, this means that the sense plate 21 and the guard ring 22 are completely enclosed within two non-conductive layers 41 and 51. It is noted that, for sake of clarity, the first conductive layer 42 is depicted over the entire extent of the sensor body 2, even in those locations where the conductive material has been removed. Thus, where the original flex foil 40 had comprised a conductive layer 42 over its entire surface, the first flex foil 40 in the sensor body 2 only has the conductive portions 21 and 22 remaining. Outside these portions 21 and 22, the layer 42 is actually not present anymore, so that the first non-conductive layer 41 and the second non-conductive layer 51 are actually attached directly to each other in those portion where the first conductive layer 42 has been removed. However, for sake of clarity, the drawing of FIG. 3 shows a distance between first non-conductive layer 41 and second non-conductive layer 51, representing the removed portions of first conductive layer 42. The same applies, mutatis mutandis, for the other layers, as should be clear to a person skilled in the art.

It is further noted that the active portions of the sensor device 1 as far as the first conductive layer 42 is concerned, are the said portions 21 and 22. The first conductive layer 42 may have been removed entirely outside these portions 21 and 22, but it is also possible that further portions of the first conductive layer 42 are still remaining, having no active function for the sensor device, having no disturbance on the functioning of the sensor device, and possibly even contributing to the shielding of outside fields, as long as such further portions of first conductive layer 42 are not in electrical contact with the portions 21 or 22.

In the second conductive layer 52 of the second flex foil 50, a guard plate 53 is defined, having, in the most preferred embodiment, an extent which at least corresponds to the extent of the guard ring 22, and may be even extending beyond the outer perimeter of the guard ring 22. Outside the guard plate 53, the second conductive layer 52 has been removed entirely in this embodiment.

The third flex foil layer 60 is attached to the second flex foil layer 50, such that the third non-conductive layer 61 of the third flex foil layer 60 is in contact with the guard plate 53; thus, the guard plate 53 is entirely embedded between non-conductive layers 51 and 61. In the third conductive layer 62 of the third flex foil 60, only a small portion around the perimeter of the sensor body 2 has been removed, so that in the first wing 3 a large portion 63 of the third conductive layer 62 remains, defining the first ground plate 13 of the first wing part 3. Likewise, a large portion 66 of the third conductive layer 62 remains in the second wing part 4, defining the second ground plate 14 of the second wing part 4.

FIG. 3 shows that the third conductive layer 62 may still be present in the fold portion 5 of the sensor body 2. Then, it is desirable that parts of the third conductive layer 62 are etched away in this folding portion 5, leaving a few small conductive lines 15 connecting the first ground plate 13 with the second ground plate 14, as shown in FIG. 1A. By removing a large part of the third conductive layer 62 in the fold portion 5, the flexibility of this fold portion 5 is improved. As long as these conductive lines 15 are intact, the contacts 11 and 12 may even be dispensed with. However, in the case of an embodiment having contacts 11 and 12 as mentioned before, the connecting lines 15 may be dispensed with, in which case the third conductive layer 62 may be removed entirely in the fold portion 5, further increasing the flexibility of the fold portion 5.

The fourth flex foil layer 70 has its fourth non-conductive layer 71 attached to the third conductive layer 62 of the third flex foil 60. The fourth conductive layer 72 of the fourth flex foil 70 defines the inner surface 6 of the sensor device 1. The fourth conductive layer 72 has been etched away over a large part, leaving the electric contacts 11 and 12, and also leaving electric circuit lines connecting the terminals of the circuit components 17 and the contact pads 16. Since the fourth conductive layer 72 has been removed over the major part of the surface of the fourth flex foil 70, one may also say that the inside surface 6 of the sensor device 1 is defined by the free surface of the fourth non-conductive layer 71, and that this inside surface 6 is provided with conductive contact portions 11 and printed circuit lines 73, 74, 75.

The guard ring 22 is electrically connected to the guard plate 53, by at least one electrical conductor 81 which crosses the second non-conductive layer 51 and which hereinafter will be indicated as an “interconnector”. In the preferred embodiment, the sensor device 1 comprises a series of such interconnectors 81, arranged in a circular pattern, at mutual intervals, which may be as small as 1-3 mm. The guard plate 53 acts as a shield against electrical fields, largely preventing such electrical fields from reaching the sense plate 21. The combination of the shield ring 22 and the array of interconnecting connectors 81 further improves the shielding effect, more or less as a Faraday's cage. The ground plate 13 of the first wing part 3, which in use will be connected to a predefined voltage level, preferably zero voltage, further helps to shield off such electrical field. It can easily be seen that, when the sensor device 1 is applied to the skin of a human body, there remains only a small gap between the ground plane 13, 63 and the outer surface 7 of the sensor device, this gap having a width defined by the combined thicknesses of the three non-conductive layers 41, 51 and 61, which will typically be less than 100 μm. Electrical field lines which are capable of penetrating this gap are further shielded by the Faraday's cage defined by guard plate 53, guard ring 22, and interconnectors 81.

In order to keep the possible influence of electrical fields from the surrounding as small as possible, an electrical circuit for processing the pick up signals is placed on the inner surface 6 of the first wing part 3, having its input terminal as close to the sense plate 21 as possible. According to an important aspect of the present invention, a small opening 54 is defined in the second conductive layer 52, for instance by etching away a corresponding small portion of the second conductive layer 52, and likewise a small opening 64 is arranged in the third conductive layer 62, these two openings 54 and 64 being aligned with each other. A first circuit portion 73 of the fourth conductive layer 72 is defined in alignment with said openings 54 and 64. FIG. 3 shows that the first circuit portion 73 and said openings 54 and 64 are aligned with the sense plate 21, and that a second interconnector 82, passing the second, third and fourth non-conductive layers 51, 61 and 71, connects the sense plate 21 to the first circuit portion 73, extending through said opening 54 and 64, such that this second interconnector 82 does not contact the guard plate 53 nor the ground plate 63. FIG. 3 also shows a circuit component 17 in the form of a package with terminal leads, an input terminal lead 17a being electrically connected to said first circuit portion 73. In the preferred embodiment, this input terminal lead 17a is substantially aligned with the second interconnector 82. The circuit component 17 shown in FIG. 3 comprises an amplifier, as will be explained later.

According to a further important aspect of the present invention, a second opening 65 is defined in the ground plate 63, and a third interconnector 83 connects a second circuit portion 74 of the fourth conductive layer 72 with the guard plate 53. This third interconnector 83 may even, as shown, extend to the guard ring 22. The third interconnector 83 thus passes the second, third and fourth non-conductive layers 51, 61 and 71, contacts the second conductive layer 52, and extends through the second opening 65 of the third conductive layer 62 such as not to make electrical contact with the third conductive layer 62. The second circuit portion 74 is connected, through a printed circuit line of the fourth conductive layer 72, to a third circuit portion 75, to which an output terminal lead 17b of the amplifier component 17 is connected.

An important feature of the interconnectors 81, 82, 83 is that they do not extend through the first non-conductive layer 41. Thus, although the first interconnector 81 makes contact with the guard ring 22 portion of first conductive layer 42, the first interconnector 81 does not extend through the first non-conductive layer 41. More particularly, the first non-conductive layer 41 always covers the first interconnector 81 in order to prevent the possibility of galvanic contact with the first interconnector 81 from the side of the outside surface 7. The same applies to the second and third interconnectors 82 and 83.

In FIG. 3, the interconnectors 81, 82, 83 are illustrated as thin, longitudinal conductors. Although such embodiment is not impossible, it is rather impractical in view of the small thickness of the flex foil layers. In a more practical, preferred embodiment, the interconnectors 81, 82, 83 are provided as metallized via's. The art of making metallized via's to provide a through-connection between two conductive layers on opposite sides of a thin non-conductive substrate is an art known per se. Nevertheless, the following figures schematically illustrate possible steps in a manufacturing process for manufacturing a sensor device according to the present invention.

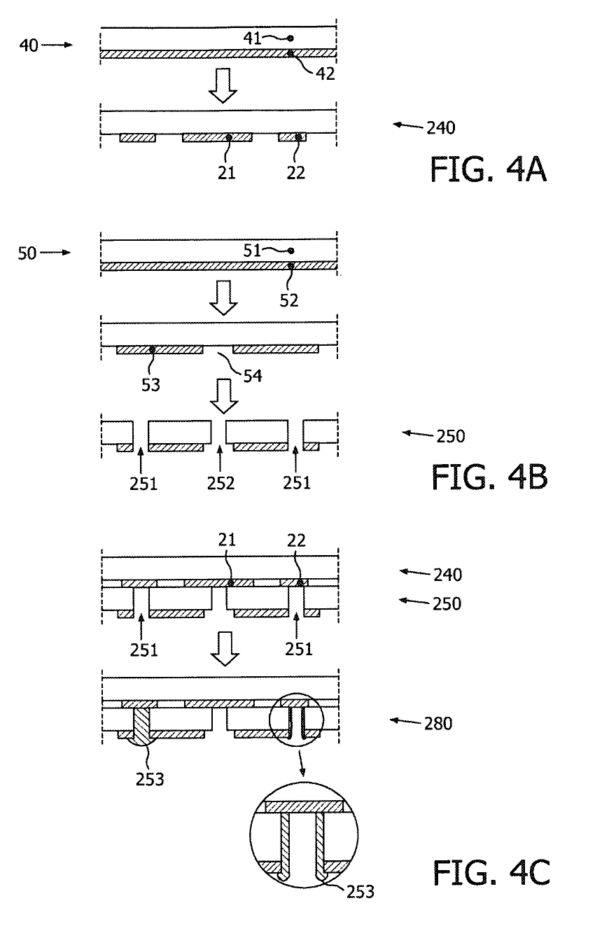

FIG. 4A schematically shows a cross section of a part of the first flex foil 40, comprising the first non-conductive layer 41 and the first conductive layer 42 extending over the entire surface. Parts of the conductive layer 42 are removed, for instance by an etching process, so that the sense plate 21 and the guard ring 22 remain. In this condition, this flex foil will be indicated as first intermediate product 240.

In a similar manner, FIG. 4B illustrates the second flex foil 50 with the second non-conductive layer 51 and the second conductive layer 52 extending over the entire surface. Parts of the conductive layer 52 are removed, so that the guard plate 53 with the opening 54 remains. In a next step, via's 251 and 252 are made, extending as through hole over the entire thickness of the second flex foil 50. A first via 251 penetrates the guard plate 53, a second via 252 is aligned with the opening 54. In this condition, the foil will be indicated as second intermediate product 250. As will become clear, the first via 251 in FIG. 4B actually represents a series of vias in a circular pattern.

In a next step, the first and second intermediate products 240 and 250 are attached onto each other, as illustrated in FIG. 4C, in such a way that the first via's 251 are aligned with the guard ring 22, while the second via 252 is aligned with the sense plate 21. The resulting product will be indicated as a stacked intermediate product 280.

In a next step, the first via's 251 are metallized. Since metallization processes are known per se, such process will not be explained here. It suffices to note that the metallization 253 in the via 251 makes electrical contact with the shield ring 22 as well as with the shield plate 53. In the left-hand side of FIG. 4C is illustrated that the metallization 253 may be provided as a solid filling of the via 251, but the right-hand side of FIG. 4C, especially the enlarged detail, illustrates that the metallization 253 may be provided as a cylindrical conductor. In both cases, the figure shows that the metallization 253 has a head portion (left hand side) or a collar portion (right hand side) extending above the free surface of the guard plate 53, but the metallization process may also be performed in such a way that the metallization 253 is flush with the free surface of the guard plate 53.

In a similar way, the third flex foil 60 may be processed to provide a third intermediate product comprised of the third non-conductive layer 61 and the ground plate 63 with openings 64 an 65, the fourth flex foil 70 may be processed to provide a fourth intermediate product comprised of the fourth non-conductive layer 71 with contacts 11 and 12 and with printed circuit portions 73, 74, 75, the fourth intermediate product 270 may be processed to provide via's aligned with the contacts 11, 12, the third and fourth intermediate products may be attached to each other, and via's may be provided in the stacked third and fourth intermediate products, extending through first contact portion 73 and first opening 64 and extending through second contact portion 74 and corresponding opening 75, over the entire thickness of the two stacked intermediate products. These steps are not individually illustrated. Then, the stacked combination of third and fourth intermediate products is attached to the stacked intermediate product 280, such that the via extending through the first contact portion 73 and corresponding opening 64 is aligned with the second via 252, and such that the second circuit portion 74 and corresponding opening 65 are aligned with the guard plate 53. It should be noted that it is now not necessary that this via is aligned with the metallized first via 251.

Then, in a next processing step, the via's are metallized. The metallization of a via extending through a contact 11 or 12 will make electrical contact with such contact 11, 12 on the one hand and with the ground plate 63 on the other hand, thus providing an interconnector 84. The metallization of the via extending through the second circuit portion 74 and the second opening 65 will make electrical contact with the second circuit portion 74 on the one hand and the guard plate 53 on the other hand, but will not make contact with the ground plate 63 in view of the relatively large opening 65. Similarly, the metallization of the via extending through the first circuit portion 73 and the first opening 64, and aligned with the second via 252, will make electrical contact with on the one hand the first circuit portion 73 and on the other hand the sense plate 21, but will not make electrical contact with the guard plate 53 nor with the ground plate 63 in view of the dimensions of the openings 54 and 64.

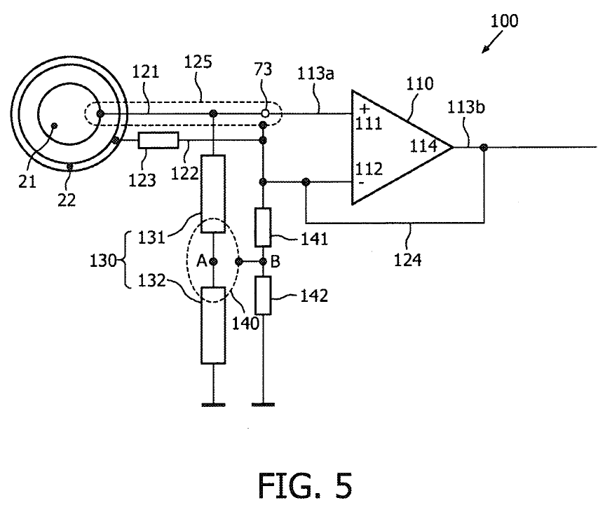

FIG. 5 is a block diagram schematically illustrating an input stage of a signal processing circuit 100 attached on the inner surface 6 of the first wing portion 3. As an important component of this processing circuit, the figure shows a differential amplifier 110, such as an operational amplifier, with a non-inverting input 111, an inverting input 112, and an output 114. This amplifier 110 is part of the component 17 shown in FIG. 3, and the non-inverting input 111 is connected to first terminal lead 17a while the output 114 is connected to second terminal lead 17b.

FIG. 5 shows that the sense plate 21 is connected to the non-inverting input 111 of the amplifier 110, through a conductor 121 which is designed to be as short as possible, and which includes the metallized via 82 and possibly a short piece of printed circuit line 73. The amplifier 110 is a type having a very high input impedance. The amplifier 110 is basically connected as a buffer amplifier, having its inverting input 112 connected to its output 114 through a line 124, so that the amplifier's output 114 carries the same voltage signal as the amplifier's input 111. The circuitry 100 may have further signal processing components, or the amplifier's output 114 may be connected straight to one of the contact pads 16, but this is not essential and not illustrated in the figures.

In use, when placed in close proximity to a person's body, the sense plate 21 has a capacitive coupling with the body, the first insulating layer 41 acting as a dielectricum. The capacitance value of this coupling is typically in the order of a few pF. The input 111 of the amplifier 110 has an input resistance which, in a suitably selected amplifier, may be approximated by infinity. However, it is desirable to provide a defined leak-resistance to zero voltage level, which is provided by the resistance 130 connected between the amplifier's input terminal 73 and ground. The combination of coupling capacity and leak-resistance forms a high-pass filter. It is desirable to have the characterizing turnover frequency of this high-pass filter as low as possibly, in the order of 0.2 Hz. This leads to a design value of 100 GΩ or higher for the resistance 130.

Apart from a capacitive coupling with the body, the sense plate 21 also has a capacitive coupling with sources of electrical voltages in the surroundings. Although this coupling has a very low capacitance value, in the order of a few fF, the voltage levels of such sources may be quite high, so that the resulting voltage induced as a result of this coupling in the sense plate 21 may typically range in the order of 100 mV. The function of the shield plate 53 located closely behind the sense plate 21, enhanced by the preferred shield ring 22 and the series of interconnectors 81 surrounding the sense plate 21, is to shield off such disturbing electrical fields, effectively reducing the coupling capacitance between the sense plate 21 and the surroundings.

It is to be noted that the sense plate 21 also has a capacitive coupling with the shield plate 53 and the shield ring 22. Any difference in voltage level between the sense plate 21 and the shield plate 53 will cause a disturbing current between the sense plate 21 and the shield plate 53, affecting the measuring signal. In order to eliminate or at least reduce this problem, the shield ring 22 and the shield plate 53 are connected to the amplifier's output 114 via a line 122, which may include a resistor 123, which may have a value in the order of a few kilo-ohms. As a result, the voltage level of the shield ring 22 and the shield plate 53 will be substantially equal to the voltage level of the amplifier's output 114, which in turn is substantially equal to the voltage level of the amplifier's input 111, hence substantially equal to the voltage level of the sense plate 21. Thus, such disturbing currents are effectively avoided. Also, disturbing currents caused by possible fouling of the interface between insulating layers 41 and 51 are likewise effectively avoided.

Although the shield plate 53 shields the sense plate 21 against outside electrical fields, the interconnector 82, the amplifier's input terminal 17a, and the printed circuit lines 73 connected to the amplifier's input terminal 17a, are all located “beyond” the shield plate 53, so they still have a capacitive coupling with the surroundings. Also, a creep current may be caused by some fouling of the inside surface 6. In order to reduce the potential problems caused by such fouling, the fourth conductive layer 72 comprises a conductive shield line 125 which surrounds all printed circuit lines 73, 121 connected to the sense plate 21, as shown in dotted lines in FIG. 5, which shield line 125 is also connected to the amplifier's output 114.

In practice, it may be difficult to find a resistor specimen having the desired resistance value of 100 GΩ, and/or such resistors are bulky and expensive. As a consequence, it may be necessary to form the leak-resistance 130 as a combination of two (or more) resistors 131, 132 in series. Then, the node A between two of those resistors 131, 132 forms a capacitive coupling with the surroundings, which, via the resistor 131, may still affect the signal at the amplifier's input 111. To reduce this effect, this node A is also surrounded by a guard ring 140, which is also connected to the amplifier's output 114, not directly, but by connecting this guard ring 140 to a node B of a series combination of two (or more) resistors 141, 142. These resistors are chosen such that the ratio of resistance values R(141)/R(142) is substantially equal to the ratio of resistance values R(131)/R(132).

To further reduce the effect of the circuit lines and circuit components being sensitive to outside electrical fields, the sensor device 1 has the second wing part 4 with the second ground plate 14, which is electrically connected to the first ground plate 13, either via one or more conductive lines 15 in the third conductive layer 62, or via the contacts 11, 12, or both. In the ready-to-use condition, when the second wing 4 is folded over the first wing 3, the second ground plate 14 extends over the circuitry 100, i.e. actually covers the circuit components 17, 110, 123, 131, 132, 141, 142, and interconnecting circuit lines 73, 74, 75, 121, 122, 124, 125, thus providing a shield against external electrical field for these components and circuit lines, which are enveloped between the two ground plates 13 and 14. In this context, it is preferred that the two ground plates 13 and 14 have their edges electrically connected together on opposite sides of the circuitry 100. For this reason, the contacts 11 are located on opposite sides of the circuitry 100.

It should be clear to a person skilled in the art that the present invention is not limited to the exemplary preferred embodiment discussed above, but that several variations and modifications are possible within the protective scope of the invention as defined in the appending claims.

For instance, instead of using two-layered flexfoils, it is possible to use a flexfoil with two conductive layers on opposite sides of a non-conductive layer, or a flexfoil with two non-conductive layers on opposite sides of a conductive layer.

Further, although in the preferred embodiment the first and second ground plates 13 and 14 are implemented as portions 63 and 66 of one and the same conductive layer 62, it is possible that the second ground plate 14 of the second wing 4 is implemented as a portion of a different conductive layer 42, 52, connected to corresponding contacts 12 via corresponding interconnectors.

Further, although in the preferred embodiment the device comprises two wing parts folded onto each other, it is also possible that the two wing parts are implemented as separate items stacked on top of each other.

Further, although in the preferred embodiment the guard plate 53 is a “solid” plate having a contour and size corresponding to the contour and size of the guard ring 22, it is possible that the guard plate is somewhat smaller, and/or that the guard plate has small interruptions, such as to have for instance a contour in the shape of spokes, without losing its functionality entirely.

1. Biometric sensor device (1), suitable for capacitively sensing bio-electrical signals, the device comprising:
a thin, flexible, layered sensor body (2) having an inner surface (6) and an outer surface (7) opposite the outer surface, the body (2) comprising a first body part (3) which comprises:
an electrically conductive sense plate (21) in a first conductive layer (42);
a first non-conductive layer (41) between the sense plate (21) and the outer surface (7);
an electrically conductive shield plate (53) in a second conductive layer (52), overlaying the sense plate (21) at the side opposite the outer surface, and having a size at least corresponding to the size of the sense plate and preferably projecting beyond the contours of the sense plate;
the shield plate (53) having a passage opening (54);
a second non-conductive layer (51) between the sense plate (21) and the shield plate (53);
electrically conductive circuit lines (73, 74, 75, 125) in a conductive circuit layer (72) on the inner surface (6);
a non-conductive separation layer (61, 71) between the shield plate (53) and the electrically conductive circuit lines (73, 74, 75);
the device further comprising:
an electronic signal processing circuit (100) having circuit components (17) mounted on the inner surface (6), the circuit (100) comprising at least one differential amplifier (110) having a first input (111) and an output (114);
a first electrically conductive interconnector (82) crossing the second non-conductive layer (51) and the separation layer (61, 71), extending through the passage opening (54) of the shield plate (53), for coupling the sense plate (21) to the first input (111) of said amplifier (110).

2. Device according to claim 1, wherein each non-conductive layer is made of capton.

3. Device according to claim 1, wherein the sensor body (2) is implemented as a stack of flexfoil layers (40; 50; 60; 70) attached to each other, each flexfoil layer comprising the combination of at least one conductive layer (42; 52; 62; 72) and at least one non-conductive layer (41; 51; 61; 71).

4. Device according to claim 1, further comprising an electrically conductive shield ring (22) around the sense plate (21) in the first conductive layer (42), the shield ring (22) being electrically connected to the shield plate (53).

5. Device according to claim 4, further comprising a series of second electrically conductive interconnectors (81) crossing the second non-conductive layer (51), each second interconnector (81) contacting the shield ring (22) and the shield plate (53).

6. (canceled)

7. Device according to claim 1, wherein the shield plate (53) is electrically connected to the output (114) of the amplifier (110).

8. Device according to claim 7, wherein the amplifier's output (114) is connected to the amplifier's inverting input (112), and wherein the sense plate (21) is connected to the amplifier's non-inverting input (111).

9. Device according to claim 7, further comprising a third electrically conductive interconnector (83) crossing the separation layer (61, 71), for coupling the shield plate (53) to the amplifier's output (114).

10. Device according to claim 1, further comprising:
an electrically conductive ground plate (13; 63) in a third conductive layer (62) between the shield plate (53) and the circuit lines (73, 74, 75), overlaying the shield plate (53) and the processing circuit (100);
a third non-conductive layer (61) between the shield plate (53) and the ground plate (13; 63);
a fourth non-conductive layer (71) between the ground plate (13; 63) and the circuit lines (73, 74, 75).

11. (canceled)

12. Device according to claim 10, wherein the ground plate (13; 63) has a first opening (64) through which the first interconnector (82) extends without making contact with the ground plate (13; 63), and wherein the ground plate (13; 63) has a second opening (65) through which the third interconnector (83) extends without making contact with the ground plate (13; 63).

13. Device according to claim 1, wherein said circuit components (17) comprise at least one mounted IC amplifier package.

14. Device according to claim 13, wherein said amplifier package has an input terminal lead (17a) aligned with said first interconnector (82).

15. Device according to claim 1, wherein said circuit components (17) comprise at least one bare IC semiconductor dye.

16. (canceled)

17. Device according to claim 1, further comprising a resistor (130) connected between the first input (111) of said amplifier (110) and a fixed voltage line, preferably a zero voltage line;
said resistor (130) having a resistance value in the order of about 1 GΩ.

18. (canceled)

19. (canceled)

20. Device according to claim 1, wherein said circuit lines (73, 74, 75, 125) comprise a shield line (125) extending as a closed loop surrounding all printed circuit lines (73, 121) which are connected to the sense plate (21), said shield line (125) being connected to the amplifier's output (114).

21. Device according to claim 1, wherein said sensor body (2) comprises two wing parts (3; 4) and a fold portion (5) connecting the two wing parts (3; 4) to each other in a foldable manner;
wherein said sense plate (21), shield plate (53), and processing circuit (100) are arranged in a first wing part (3);
and wherein a second wing part (4) comprises a second electrically conductive ground plate (14; 66).

22. (canceled)

23. Device according to claim 21, further comprising:
an electrically conductive first ground plate (13; 63) in a third conductive layer (62) between the shield plate (53) and the circuit lines (73, 74, 75), overlaying the shield plate (53) and the processing circuit (100);
a third non-conductive layer (61) between the shield plate (53) and the first ground plate (13; 63);
a fourth non-conductive layer (71) between the first ground plate (13; 63) and the circuit lines (73, 74, 75);
wherein the second ground plate (14; 66) is electrically connected to the first ground plate (13; 63).

24. Device according to claim 23, wherein the second ground plate (14; 66) and the first ground plate (13; 63) are implemented as portions of one and the same layer (62).

25. Device according to claim 24, further comprising at least one connecting line (15) in the fold portion (5), implemented as a portion of the same layer (62), connecting the second ground plate (14; 66) to the first ground plate (13; 63).

26. Device according to claim 23, further comprising:
in the first wing part (3), at least one first contact (11) implemented as a portion of the conductive circuit layer (72) on the inner surface (6), the first contact (11) being electrically connected to the first ground plate (13; 63);
in the second wing part (4), at least one second contact (12) implemented as a portion of the conductive circuit layer (72) on the inner surface (6), the second contact (12) being electrically connected to the second ground plate (14; 66);
wherein said first and second contacts (11; 12) are positioned such that, when the body (2) is folded at the fold portion (5), said first and second contacts (11; 12) are substantially aligned with each other.

27. (canceled)

28. Device according to claim 21, wherein said two wing parts (3, 4) are folded together and are attached to each other.

29. (canceled)

30. Device according to claim 1, wherein said sensor body (2) further comprises a second body part (4) overlying the first body part (3), the second body part (4) comprising:
an electrically conductive second ground plate (14; 66) overlying the processing circuit (100) at the side opposite to the shield plate (53);
at least one insulating layer (71) between the ground plate (14; 66) and the processing circuit (100);
at least one insulating layer (41, 51, 61) between the ground plate (14; 66) and the outer surface (7) of the second body part (4).

31. Device according to claim 30, further comprising:
an electrically conductive first ground plate (13; 63) in a third conductive layer (62) between the shield plate (53) and the circuit lines (73, 74, 75), overlaying the shield plate (53) and the processing circuit (100);
a third non-conductive layer (61) between the shield plate (53) and the first ground plate (13; 63);
a fourth non-conductive layer (71) between the first ground plate (13; 63) and the circuit lines (73, 74, 75);
wherein the second ground plate (14; 66) is electrically connected to the first ground plate (13; 63).

32. (canceled)

33. (canceled)

34. (canceled)

35. (canceled)

36. Device according to claim 1, incorporated in clothing.

37. Device according to claim 1, incorporated in surface material of an object to be contacted by a person, the object selected from consisting of a chair, a bed, an examination table, a saddle, a steering wheel, and a baby's incubator.
